(19) United States
(12) Patent Application Publication
(10) Pub. No.: US 2022/0306983 A1
(43) Pub. Date: Sep. 29, 2022

(54) A LACTOBACILLUS PLANTARUM STRAIN AR113 WITH PROTECTION EFFECT ON APOPLEXY

(71) Applicant: UNIVERSITY OF SHANGHAI FOR SCIENCE AND TECHNOLOGY, SHANGHAI (CN)

(72) Inventors: LIANZHONG AI, SHANGHAI (CN); ZHIQIANG XIONG, SHANGHAI (CN); YONGJUN XIA, SHANGHAI (CN); GUANGQIANG WANG, SHANGHAI (CN); HUI ZHANG, SHANGHAI (CN); FENGXI LAI, SHANGHAI (CN); XIN SONG, SHANGHAI (CN)

(21) Appl. No.: 17/431,144

(22) PCT Filed: Sep. 11, 2020
(86) PCT No.: PCT/CN2020/114637

§ 371 Date: Aug. 13, 2021

(30) Foreign Application Priority Data
Mar. 2, 2020 (CN) 202010135379.2

(51) Int. Cl. C12N 1/20; A61K 35/747; A61P 9/10

(52) U.S. Cl. CPC C12N 1/205; A61K 35/747; A61P 9/10; C12R 2001/25

(57) ABSTRACT

A Lactobacillus plantarum strain AR113 with protection effect on apoplexy. The Lactobacillus plantarum strain AR113 deposited in China General Microbiological Culture Collection Center (CGMCC) under CGMCC Accession No. 13909 on Mar. 22, 2017. The Lactobacillus plantarum AR113 strain can increase the antioxidant enzyme activity of brain tissue, reduce the level of oxidation products, activate the Nrf-ARE signal pathway, and regulate the relative expression of antioxidant factors Nrf2, NQO-1, and HO-1. Additionally, it can down-regulate the mRNA expression of pro-apoptotic factors Cyt-C, Caspase-3 and Bax, and up-regulate the relative expression of anti-apoptotic factor Bcl-2, thereby improving brain cell damage caused by cerebral ischemia and reperfusion.

### RELATED APPLICATIONS

This application is a § 371 application of PCT/CN2020/114637 filed Sep. 11, 2020, which claims priority from Chinese Patent Application No. 202010135379.2 filed Mar. 2, 2020, each of which is incorporated herein by reference in its entirety.

### FIELD OF THE INVENTION

The present invention belongs to the microbiological technology field, in particular relates to a Lactobacillus plantarum strain AR113 with protection effect on apoplexy.

### BACKGROUND OF THE INVENTION

Apoplexy is a serious manifestation of a variety of cerebrovascular diseases, including hemorrhagic and ischemic apoplexy. In China, the incidence of ischemic apoplexy is much higher than that of hemorrhagic apoplexy, accounting for about 70% of the total. After an ischemic apoplexy, restoring cerebral blood flow as soon as possible is one of the most effective medical methods to treat patients. However, blood flow reperfusion will aggravate cell damage and cause aggravation of the disease, that is, ischemia reperfusion injury (I/R). Inflammation, oxidative stress, and cell apoptosis are the main mechanisms of cerebral ischemia-reperfusion injury, and they are also important causes of neurological deficits in post-apoplexy patients.

As a kind of active microorganisms beneficial to the host, probiotics have been widely used in the adjuvant treatment of intestinal diseases. The probiotic functions and mechanisms of probiotics such as anti-inflammatory, antioxidant, immune enhancement and micro-ecology regulation have been confirmed in various experiments. Due to the existence of the flora-gut-brain axis, the application of probiotics to treat nervous system diseases has also been obtained. More and more attention is paid, and whether probiotics can reduce cerebral ischemia-reperfusion injury and its possible mechanism are worth exploring. Both in vivo and in vitro experiments have proved that the strain Lactobacillus plantarum AR113 preserved in our laboratory has strong antioxidant capacity and can effectively alleviate the damage caused by oxidative stress [1,2], and AR113 has good basic characteristics for intraoral application and has the potential to be used as intraoral probiotics.

### REFERENCES

[1] Lin X, Xia Y, Wang G, et al. Lactobacillus plantarum AR501 Alleviates the Oxidative Stress of D-Galactose-Induced Aging Mice Liver by Upregulation of Nrf2-Mediated Antioxidant Enzyme Expression[J]. Journal of Food Science, 2018, 83(7): 1990-1998.

[2] Lin X, Xia Y, Wang G, et al. Lactic Acid Bacteria With Antioxidant Activities Alleviating Oxidized Oil Induced Hepatic Injury in Mice[J]. Frontiers in Microbiology, 2018, 9: 2684.

### SUMMARY OF THE INVENTION

The technical problem to be solved by the present invention is to provide a Lactobacillus plantarum with protection effect on apoplexy.

The present invention provides a Lactobacillus plantarum AR113 strain with protection effect on apoplexy, characterized by that the Lactobacillus plantarum AR113 strain was deposited in the China General Microbiological Culture Collection Center (CGMCC) under CGMCC Accession No. 13909 on Mar. 22, 2017.

The invention also provides an application in preparing products with protection effect on apoplexy by using the Lactobacillus plantarum AR113 strain.

When applied in the intraoral cavity of I/R rats, the Lactobacillus plantarum AR113 of the present invention has a regulating effect on its tongue bacterial flora and intestinal flora, and has a certain protective effect on neurological deficits and brain damage. The main reason is that AR113 has excellent anti-oxidant and anti-apoptotic activity. Intraoral application of AR113 can regulate the imbalance of the tongue bacterial flora caused by I/R injury, and it can maintain the steady state of the intestinal flora. At the same time, AR113 can alleviate neurological deficits and brain cell oxidative stress damage caused by I/R injury, and has a certain improving effect on apoptosis.

### BRIEF DESCRIPTION OF THE DRAWINGS

FIG. 1 is the schematic diagram of animal experiment design in embodiment 1.

FIG. 2 is the schematic diagram of horizontal composition of rat tongue bacterial flora in embodiment 2.

FIG. 3 is the cluster diagram of the horizontal species abundance of rat tongue bacterial families in embodiment 2.

FIG. 4 is the schematic diagram of non-metric multidimensional scale analysis of rat tongue bacterial flora in embodiment 2.

FIG. 5 is the diagram of main species differences among rat tongue bacteria flora groups in embodiment 2.

FIG. 6 is the composition diagram of the genus level of intestinal flora of rats in embodiment 3.

FIG. 7 is the schematic diagram of main species difference analysis in rat intestinal bacterial genus level in embodiment 3.

FIG. 8 is the schematic diagram of non-metric multidimensional scale analysis of rat intestinal flora in embodiment 3.

FIG. 9 is the schematic diagram of LEfSe analysis among rat intestinal bacterial groups in embodiment 3.

FIG. 10 is the schematic diagram of the effect of AR113 on the neurological function score of I/R rats in embodiment 4.

FIG. 11 is the schematic diagram of the movement track of the water maze experiment in embodiment 5.

FIG. 12 is the diagram of the effect of AR113 on cerebral infarction volume in I/R rats in embodiment 6.

FIG. 13 is the diagram of HE-stained brain tissues in embodiment 7.

FIG. 14 is the schematic diagram of the effect of AR113 on the levels of SOD, GSH-Px, CAT enzyme activity, MDA and H₂O₂ in the brain tissue of I/R rats in embodiment 8.

FIG. 15 is the schematic diagram of the regulatory effect of AR113 on the antioxidant-related factors in the brain tissues of I/R rats in embodiment 8.

FIG. 16 is the schematic diagram of the effect of AR113 on brain cell apoptosis in I/R rats in embodiment 9.

### DETAILED DESCRIPTION OF THE INVENTION

The present invention is further described in detail below with reference to specific embodiments and figures for a better understanding of the technical means and effects. The following embodiments are preferred cases of the present invention. And the present invention is not only limited to these embodiments.

The main reagents involved in the following embodiments such as chloral hydrate(TCA), 2,3,5-chlorotriphenyltetrazolium(TTC) and 4% paraformaldehyde were purchased from Sinopharm Chemical Reagent Co., Ltd.; BCA protein Test kit, SOD test kit, GSH-Px test kit, CAT test kit, H₂O₂ test kit and MDA test kit were purchased from Nanjing Jiancheng Bioengineering Institute; Trizol, reverse transcription kit and SYBR Green reagent kit were purchased from Takara Bio Inc. The specific DNA sequence information of the primers used in the embodiments is shown in Table 1, synthesized by Sangon Biotech (Shanghai) Co., Ltd. The bacterial diversity was sequenced by Shanghai Majorbio Bio-Pharm Technology Co., Ltd.

TABLE 1
Fluorescence quantitative q-PCR reaction
amplification primer sequence

| | Forward primer | Reverse |
|---|---|---|
| Gene | (5′-3′) | primer (5′-3′) |
| SOD | GTAGGGCCTGTCCGATGATG | CGCTACTGAGAAAGGTGCCA |
| GSH-Px | GTCCACCGTGTATGCCTTCT | CGTTCATCTCGGTGTAGTCC |
| | C | C |
| CAT | TTGTTCAGTGACCGAGGGAT | TTCCTGAGCAAGCCTTCCTG |
| | T | |
| Nrf2 | AAGCAAGAAGCCAGATACA | TCACATCACAGTAGGAAGTT |
| NQ01 | CAGCCAATCAGCGTTCGGTA | CTTCATGGCGTAGTTGAATG |
| | | ATGT |
| HO-1 | TGCAGGTGATGCTGACAGAG | GGGATGAGCTAGTGCTGATC |
| | G | TGG |
| Cyt | CCTTTGTGGTGTTGACCAGC | CCATGGAGGTTTGGTCCAGT |
| Caspase-3 | AGCTGGACTGCGGTATTGAG | GGGTGCGGTAGAGTAAGCAT |
| Bax | TTGCTACAGGGTTTCATCCA | TGTTGTTGTCCAGTTCATCG |
| | G | |
| Bcl-2 | AGCCTGAGAGCAACCGAAC | AGCGACGAGAGAAGTCATCC |
| β-actin | GGCTGTATTCCCCTCCATCG | CCAGTTGGTAACAATGCCAT |
| | | GT |

The experimental methods used in the following embodiments are conventional methods unless otherwise specified; the materials and reagents used can be obtained from commercial sources unless otherwise specified.

### Embodiment 1: Establishment of Rat Cerebral Ischemia-Reperfusion Model and Experimental Grouping

Establishment of rat cerebral ischemia-reperfusion model: All rats were fasted for 12 hours but not water-deprived before operation. After weighing, inject the rats intraperitoneally with 10% chloral hydrate (mass concentration) for anesthesia (3.5 mL/kg). Then fix the rats in the supine position, incise in the midline of the necks, and separate the muscles and fascia along the inner edge of sternocleidomastoid. Separate the left common carotid artery (CCA), external carotid artery (ECA), and internal carotid artery (ICA). Hang threads at the distal and proximal ends of the CCA and the ECA for further use. Clamp the ICA temporarily using a micro artery clamp. And then ligate the CCA and ECA proximally. Sequentially, cut a small cut 4 mm from the bifurcation of the CCA, and insert a suture into the ICA. Then tie the suture gently with a thin thread around the distal end of the CCA. After 2 hours of ischemia, pull out the suture slowly and prepare the cerebral ischemia-reperfusion model using reperfusion.

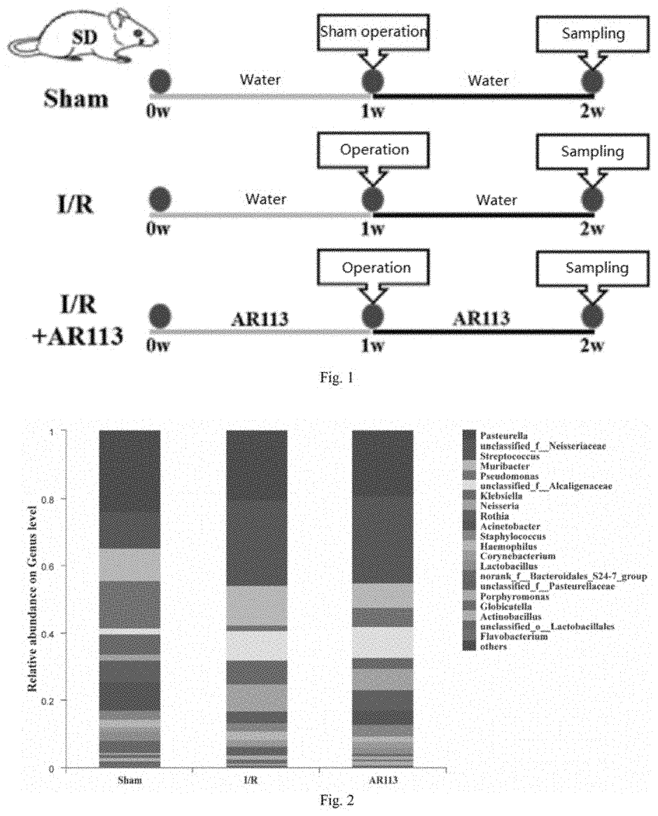

FIG. 1 shows the schematic diagram of the animal experiment design in embodiment 1 of the present invention.

After adaptive feeding for one week, divide 27 experimental rats randomly into 3 groups, each with 9 rats. The experimental design is shown in FIG. 1. Sham operation group (Sham group): separate the neck blood vessels only, no suturing step performed, and spray 1 ml of 0.01 M PBS solution every day during the experiment for 14 consecutive days. Ischemia-reperfusion group (I/R group): At the time point \textcircled{2}, i.e., the 7th day, prepare the ischemia-reperfusion model using middle cerebral artery embolization, and spray 1 ml of 0.01 M PBS solution every day for 14 consecutive days. Lactobacillus plantarum AR113 intervention group (AR113 group): spray 1 ml of AR113 bacterial suspension (109 CFU/ml) every day one week before modeling (period \textcircled{1}), and spray AR113 for one week after modeling, the same as \textcircled{1}.

### Embodiment 2: The Structural Regulation of Lactobacillus plantarum AR113 on the Tongue Coating Microbiota of I/R Rats

Detect the effect of Lactobacillus plantarum AR113 on I/R rat tongue bacterial flora using 16S rDNA high-throughput sequencing technology. The results of a diversity index change are shown in Table 2. Compared with the sham operation group, the richness and diversity of the tongue bacterial flora of I/R rats showed a downward trend, and the reduction of Chao index and Shannon index was statistically significant (P<0.05), indicating that cerebral ischemia-reperfusion injury led to the structure migration of rat tongue bacterial flora, and the abundance and diversity of the bacterial flora was significantly reduced. The intervention of AR113 significantly improved the diversity of the bacterial flora. Compared with the I/R group, after the AR113 treatment, although there was no significant difference in the abundance and diversity index of the rat tongue bacterial flora, both it showed a certain improvement effect, and it improved the diversity of I/R rat tongue bacterial flora to varying degrees.

TABLE 2
α diversity index of rat tongue bacterial flora

| Group | ACE | Chao | Shannon | Simpson | Coverage (%) |
|---|---|---|---|---|---|
| Sham | 377.9 ± 95.73 | 379.31 ± 101.77 | 3.33 ± 0.53 | 0.08 ± 0.03 | 99.90 ± 0.03 |
| I/R | 288.29 ± 87.62 | 251.76 ± 73.26* | 2.67 ± 0.38* | 0.13 ± 0.08 | 99.88 ± 0.04 |
| AR113 | 333.92 ± 44.59 | 308.1 ± 43.71 | 2.79 ± 0.37 | 0.13 ± 0.06 | 99.86 ± 0.03 |

Note:
*P < 0.05 vs Sham group.

FIG. 2 is the schematic diagram of horizontal composition of rat tongue bacterial flora in embodiment 2.

As shown in FIG. 2, 13 bacteria genera account for more than 80% of the species abundance, and are the main bacteria genera of rat tongue fur, including Pasteurella, unclassified_f_Neisseriaeae, Streptococcus, Muribacter, Pseudomonas, unclassified_f_Alcaligenaceae, Klebsiella, Neisseria, Rothia, Acinetobacter, Staphylococcus, Haemophilus and Corynebacterium.

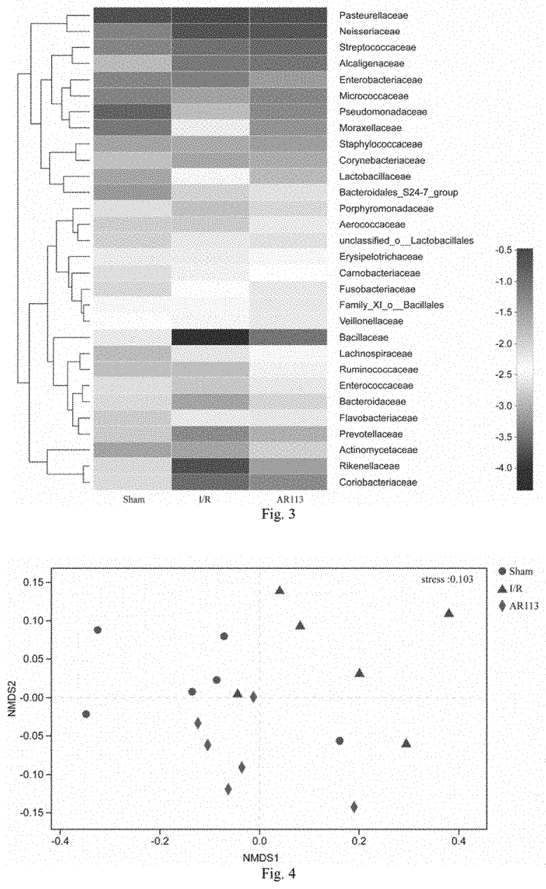

FIG. 3 is the cluster diagram of the horizontal species abundance of rat tongue bacterial families in embodiment 2.

Perform species abundance cluster analysis at the family level, select the top 30 species in relative abundance, cluster the species and draw a heat map to show the species composition information of the community. The color changes reflect the difference in abundance of different species at the family level between groups. The relative abundance of species in the rat tongue bacterial flora of the I/R group was lower than that of the Sham group, and the species abundance of the AR113 group was higher than that of the I/R group to a certain extent. The species include Moraxellaceae, Lactobacillaceae, Bacteroidales_S24-7_group, Bacillaceae, Lachnospiraceae, Ruminococcaceae, Enterococcaceae, Bacteroidaceae, Flavobacteriaceae and Prevotellaceae. Among them, the abundance of Moraxellaceae and Lactobacillaceae increased significantly, as shown in FIG. 3. The results showed that the intervention of AR113 can adjust the structure of the tongue bacterial flora of rats with cerebral ischemia-reperfusion injury, and can improve the imbalance of the tongue bacterial flora after I/R to a certain extent.

FIG. 4 is the schematic diagram of non-metric multidimensional scale analysis of rat tongue bacterial flora in embodiment 2.

In order to study the similarity of the bacterial flora structure between different samples, non-metric multidimensional scaling analysis was performed on the bacteria in the tongue coating of I/R rats. The result is shown in FIG. 4. The points of the same color and shape in FIG. 4 are the same group of samples. The closer the two sample points are, the more similar the species composition of the two samples. Stress is 0.103, and it is generally considered that when stress is less than 0.2, the graph has a certain explanatory significance, which can accurately reflect the degree of difference between samples. The samples of the I/R group are far away from the Sham group, indicating that the bacterial flora of the I/R group is different from that of the Sham group. While the AR113 group samples and the Sham group samples are concentrated in the second and third quadrants, and the distances between the points are close, indicating that the structures of the bacterial flora of the AR113 group samples and the Sham group samples are similar. The intervention of AR113 is beneficial for rebuilding the tongue bacterial flora of I/R rats and promotes the restoration of the micro-ecological balance of tongue coating.

In order to find out the main species that caused the differences in bacterial community structure, the Kruskal-Wallis rank sum test is used to perform hypothesis testing on the species abundance data between groups to obtain the P value. The analysis of differences between Sham-I/R group and I/R-AR113 group is performed on the family level, as shown in Table 3. It is found that the abundance of Neisseriaceae and Streptococcaceae increased significantly in the I/R group, while the abundances of Bacteroidales_S24-7_group, Lactobacillaceae, Lachnospiraceae, and Prevotellaceae all decreased in the I/R group, indicating that the composition and abundance of the tongue bacterial flora of rats changed statistically after cerebral ischemia and reperfusion. Thus, increasing the abundance of species including Lactobacillaceae, Aeromonadaceae, Bacillaceae and etc. may be the main mode of action for AR113 to regulate the tongue bacterial flora disorder of the I/R-injured rats.

TABLE 3
Rat tongue bacterial flora species with abundance differences at the family level

| | Sham | SD | I/R | SD | P |
|---|---|---|---|---|---|
| Taxa | (%) | (%) | (%) | (%) | value |
| Neisseriaceae | 6.57 | 4.896 | 22.68 | 17.72 | 0.04533 |
| Streptococcaceae | 6.131 | 2.428 | 10.62 | 5.192 | 0.03064 |
| Bacteroidales_S24- | 3.675 | 5.008 | 0.1772 | 0.1965 | 0.02024 |
| 7_group | | | | | |
| Lactobacillaceae | 2.754 | 3.252 | 0.3299 | 0.1808 | 0.02024 |
| Lachnospiraceae | 1.645 | 1.239 | 0.2435 | 0.2233 | 0.03064 |
| Ruminococcaceae | 1.483 | 1.44 | 0.1252 | 0.1465 | 0.02024 |
| Prevotellaceae | 1.159 | 1.161 | 0.05016 | 0.05934 | 0.01937 |
| Enterococcaceae | 0.8047 | 0.7216 | 0.1464 | 0.1101 | 0.005075 |
| Erysipelotrichaceae | 0.5894 | 0.166 | 0.272 | 0.1263 | 0.005075 |
| Coriobacteriaceae | 0.1999 | 0.138 | 0.02746 | 0.03867 | 0.03035 |
| Desulfovibrionaceae | 0.205 | 0.1728 | 0.006224 | 0.00612 | 0.03035 |
| Peptostreptococcaceae | 0.171 | 0.142 | 0.01831 | 0.0182 | 0.008239 |
| Christensenellaceae | 0.1128 | 0.1061 | 0.008055 | 0.01134 | 0.01014 |
| | I/R | SD | AR113 | SD | P |
| Taxa | (%) | (%) | (%) | (%) | value |
| Lactobacillaceae | 0.3299 | 0.1808 | 1.77 | 1.446 | 0.005075 |
| Aeromonadaceae | 0.005126 | 0.00432 | 0.03698 | 0.03306 | 0.02372 |
| Bacillaceae | 0.003295 | 0.00361 | 0.03258 | 0.02436 | 0.01916 |
| Comamonadaceae | 0.005858 | 0.007039 | 0.02599 | 0.01635 | 0.03671 |
| Sphingobacteriaceae | 0 | 0 | 0.01428 | 0.01988 | 0.02844 |
| norank_c_Actinobacteria | 0.0003661 | 0.0008968 | 0.004393 | 0.00555 | 0.02922 |
| Xanthobacteraceae | 0 | 0 | 0.004759 | 0.00469 | 0.009465 |

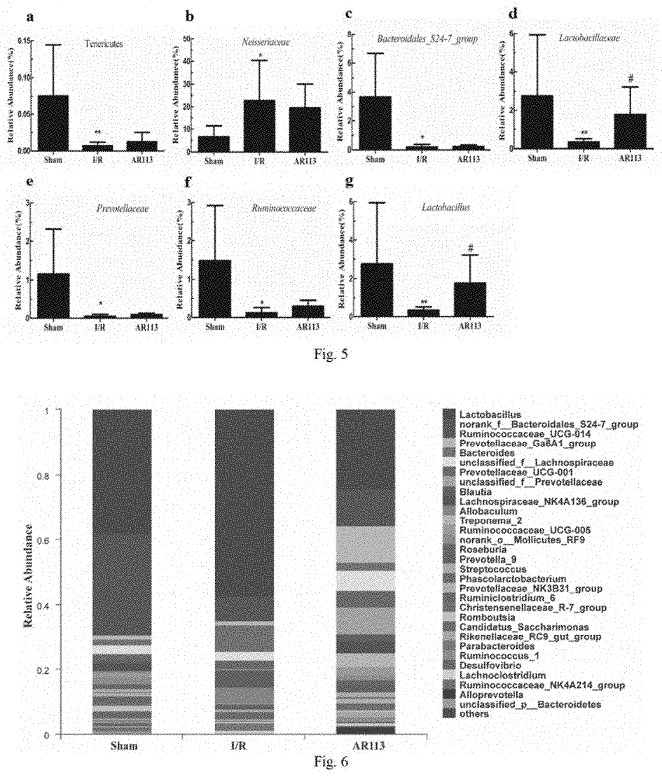

FIG. 5 is the diagram of main species differences among rat tongue bacteria flora groups in embodiment 2, in which *P<0.05, **P<0.01 vs Sham group; #P<0.05 vs I/R group. FIG. 5(a) is a comparison diagram of the genus abundance of Tenericutes. FIG. 5(b) is a comparison of the genus abundance of Neisseriaceae. FIG. 5(c) is a comparison diagram of the genus abundance of Bacteroidales_S24-7. FIG. 5(d) is a comparison diagram of the genus abundance of Lactobacillaceae. FIG. 5(e) is a comparison diagram of the genus abundance of Prevotellaceae. FIG. 5(f) is a comparison diagram of the genus abundance of Ruminococcaceae. And FIG. 5(g) is a comparison diagram of the genus abundance of Lactobacillus.

Select the species that have an influential effect in the three groups and have obvious changes in abundance to further draw a bar graph to analyze the significance of the groups, as shown in FIG. 5. At the gate level, the abundance of Tenericutes in the I/R group was significantly reduced (P<0.01), and that in the AR113 group was increased, compared with the I/R group. But there was no significant difference in the increase. At the family level, I/R damage significantly reduced the abundances of Bacteroidales_S24-7_group, Lactobacillaceae, Prevotellaceae and Ruminococcaceae, while the abundance of Neisseriaceae increased significantly. The result is basically consistent with the changes in the structure of the tongue bacterial flora of apoplexy patients in Chapter 3, in which the decrease of species abundances of Prevotellaceae, Ruminococcaceae and etc. is a common feature for brain injury. The change of tongue bacterial flora in I/R rats once again confirmed the imbalance of tongue bacterial flora after brain injury. At the genus level, the effect of AR113 can significantly increase the abundance of Lactobacillus, which was decreased due to I/R damage (P<0.05). The analysis results of the bacterial flora difference fully showed that AR113 can regulate the tongue bacterial flora of I/R rats and alleviate the imbalance of the flora caused by I/R injury.

### Embodiment 3: Structural Regulation of Lactobacillus plantarum AR113 on the Intestinal Microbiota of I/R Rats

In this embodiment, the effect of AR113 intake on the intestinal flora in stool samples of I/R rats was studied. The change of a diversity index is shown in Table 4. Compared with sham operation, the richness index (ACE, Chao) and diversity index (Shannon, Simpson) of the intestinal flora of I/R rats were significantly reduced, indicating that the structural diversity of intestinal flora of rats after I/R injury decreases. The intervention of AR113 can significantly improve the decrease of intestinal flora diversity caused by I/R injury, indicating that AR113 can regulate the intestinal flora of I/R rats to a certain extent, improving the intestinal flora.

TABLE 4
α diversity index of rat intestinal flora

| Group | ACE | Chao | Shannon | Simpson | Coverage (%) |
|---|---|---|---|---|---|
| Sham | 574.51 ± 29.60 | 592.73 ± 30.40 | 4.22 ± 0.46 | 0.05 ± 0.03 | 99.80 ± 0.01 |
| I/R | 372.56 ± 51.13*** | 386.00 ± 54.89*** | 3.24 ± 0.56** | 0.12 ± 0.05* | 99.85 ± 0.02 |
| AR113 | 571.66 ± 33.61### | 596.72 ± 30.89### | 4.08 ± 0.21## | 0.05 ± 0.02# | 99.79 ± 0.01 |

Note:
*P < 0.05, **P < 0.01, ***P < 0.001 vs Sham group;
#P < 0.05, ##P < 0.01, ###P < 0.001 vs I/R group.

FIG. 6 is the composition diagram of the genus level of intestinal flora of rats in embodiment 3.

Species including Lactobacillus, norank_f_Bacteroidales_S24-7_group, Ruminococcaceae_UCG-014, Prevotellaceae_Ga6A1_group, Bacteroides, unclassified_f_Lachnospiraceae, Prevotellaceae_UCG-001 and etc. are the main components of the rat intestinal flora. Among them, Lactobacillus is the most important genus of the three groups of rats, with the highest relative abundance in the samples. It is significantly enriched especially in I/R rats, with an abundance as high as 52.60%. However, the abundances were lower in the sham operation and AR113 groups, which were 26.16% and 12.33%, respectively. The increased abundance of Lactobacillus may play a role in regulating the autoimmune system.

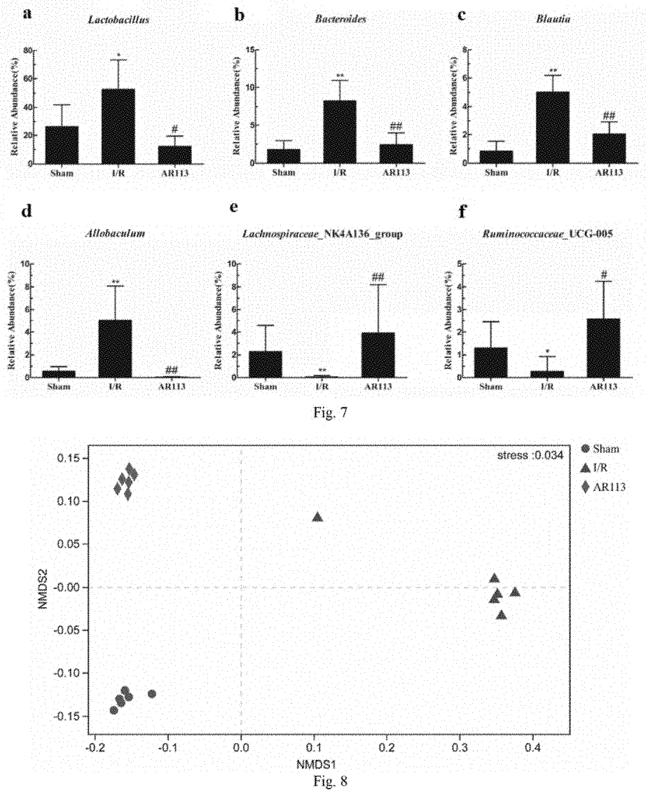

FIG. 7 is the schematic diagram of main species difference analysis in rat intestinal bacterial genus level in embodiment 3.

FIG. 7(a) is a comparison diagram of the genus abundance of Lactobacillus. FIG. 7(b) is a comparison diagram of the genus abundance of Bacteroides. FIG. 7(c) is a comparison diagram of the genus abundance of Blautia. FIG. 7(d) is a comparison diagram of the genus abundance of Allobaculum. FIG. 7(e) is a comparison diagram of the genus abundance of Lachnospiraceae_NK4A136_group. FIG. 7(f) is a comparison diagram of the genus abundance of Ruminococcaceae_UCG-005.

In order to study the intestinal flora species with significant differences between the groups, the relative abundance of the three groups of rat species was compared at the genus level. The result is shown in FIG. 7. Compared with the sham operation group, the abundances of Lactobacillus, Bacteroides, Blautia, and Allobaculum in the intestinal flora of I/R rats were significantly increased, while the abundances of Lachnospiraceae_NK4A136_group and Ruminococcaceae_UCG-005 showed a significant decline. Lachnospiraceae and Ruminococcaceae are the symbolic species of healthy intestines. After the action of AR113, it can regulate the imbalance of intestinal flora caused by I/R injury. Reducing the abundances of Lactobacillus, Bacteroides, Blautia, Allobaculum, and increasing the abundances of Lachnospiraceae_NK4A136_group and Ruminococcaceae_UCG-005 are the main action modes of AR113 in regulating the intestinal flora disorder of mice with brain injury caused by I/R.

FIG. 8 is the schematic diagram of non-metric multidimensional scale analysis of rat intestinal flora in embodiment 3.

The β diversity analysis of rat intestinal flora is performed to calculate the species information of the intestinal flora of rat samples, using non-metric multidimensional scale analysis and the Unweighted Unifrac algorithm. Compared with the tongue coating microorganisms, the intestinal microbes have higher similarity within the group. The Stress value is 0.034, which is less than 0.05, indicating that the degree of divergence of the given samples is representative and can be accurately reflected in the figure. After excluding individual samples in the I/R group, the three groups of sample points are arranged in a concentrated manner, and they are all well grouped into three categories. Different treatments will result in completely different structural compositions of the intestinal flora of rats, as shown in FIG. 8. The sample points of the I/R group are far away from the sample points of sham operation and AR113 group, indicating that the intestinal flora of rats has changed significantly after I/R injury. The sample points of the AR113 group and the sham operation group are respectively distributed in the second and third quadrants. There are differences in the structure of the flora, but they are all located in the left half of the graph, indicating that the two groups have a certain similarity. NMDS analysis results show that the intestinal flora of rats is abnormally changed after I/R injury, and the action of AR113 can regulate the imbalanced intestinal flora, making it closer to the sham operation group samples, and has a certain restorative effect on the structure of the flora.

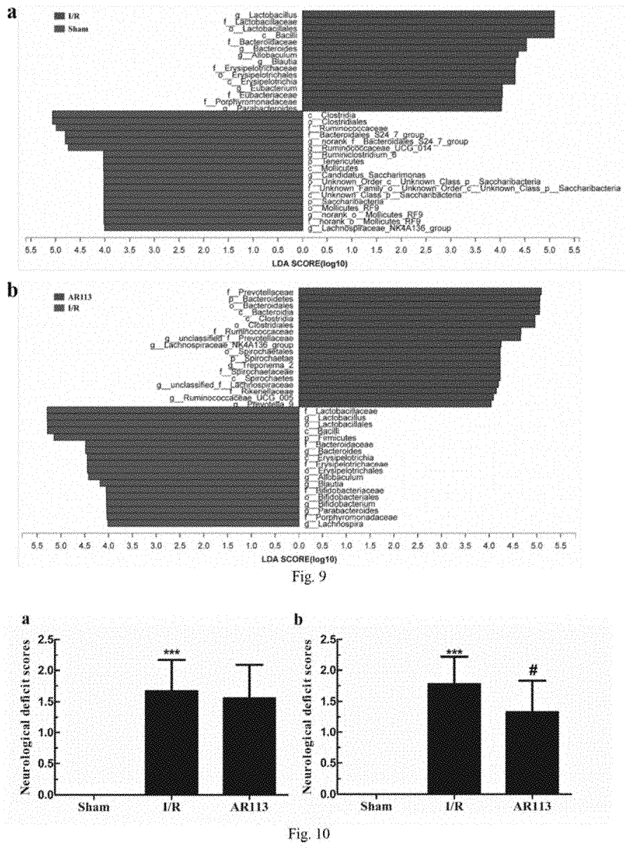

FIG. 9 is the schematic diagram of LEfSe analysis among rat intestinal bacterial groups in embodiment 3. FIG. 9(a) is a schematic diagram of the comparative analysis between the sham operation group and the I/R group. FIG. 9(b) is a schematic diagram of the comparative analysis between AR113 group and I/R group.

Perform LefSe analysis on the three groups. And as shown in the LDA distribution histogram, compared with Sham group, in I/R group, genus abundances of Lactobacillus, class Bacilli, order Lactobacillales, family Lactobacillaceae, Bacteroides, family Bacteroidaceae, Allobaculum, Blautia and Eubacterium were increased. Thus they are I/R-related intestinal species, as shown in FIG. 9(a). AR113 can increase the abundances of phylum Bacteroidetes, phylum Spirochaetae, family Prevotellaceae, family Ruminococcaceae, family Spirochaetaceae, Rikenellaceae, Prevotella_9 and Lachnospiraceae_NK4A136_group, as shown in FIG. 9(b). The results of this study show that the intervention of probiotic AR113 can significantly increase the abundances of health-related species and improve the changes of animal intestinal flora in disease states. Summing up the changes of rat tongue bacterial flora and intestinal flora, it is found that the cerebral ischemia-reperfusion (I/R) injury results to the disorder of rat tongue bacterial flora and intestinal flora. The intraoral application of AR113 can not only regulate the structure of tongue bacterial flora, but also promote the balance of intestinal microbes of rats.

### Embodiment 4: Effect of Lactobacillus plantarum AR113 on Neurological Function Score of I/R Rats

The behavioral experiment was measured in two times, and the neurological deficit was scored according to the Longa scoring standard in Table 5. The first measurement was on the day after the modeling, and the purpose was to identify whether the modeling was successful (1-3 points into the group) and to eliminate the rats that failed the modeling. The neurological deficits of the four groups of rats were measured again 7 days after the modeling. The neurological deficits are scored using a double-blind method. Three testers perform the measurements independently, and the results are averaged.

TABLE 5
Longa scoring criteria

| Behavioral experiment | Score |
|---|---|
| Normal, no neurological deficit | 0 |
| Either front paw cannot be fully extended | 1 |
| Rotating toward the paralyzed side while walking | 2 |
| Falling down toward the paralyzed side while | 3 |
| walking | |
| Can't stand and walk spontaneously, low level of | 4 |
| consciousness | |

FIG. 10 is the schematic diagram of the effect of AR113 on the neurological function score of I/R rats in embodiment 4. FIG. 10(a) is a schematic diagram of the effect of AR113 on the neurological function score of I/R rats 24 h after the modeling. FIG. 10(b) is a schematic diagram of the effect AR113 on the neurological function score of I/R rats 7 days after the modeling.

Behavioral testing is a common method to evaluate the neurological dysfunction of cerebral ischemia injury. According to the Longa scoring standard, the results of neurological deficits in each group of rats were determined. As shown in FIG. 10(a), the 24-hour time point after modeling shows that the neurological function score of the model group was significantly higher than that of the sham operation group (P<0.001), indicating that the modeling of MCAO-induced I/R rats was successful, while the administration of AR113 for one week before modeling did not significantly improve the neurological deficits of the I/R rats. The 7-day time point after modeling shows that intraoral intervention of AR113 for 14 consecutive days can alleviate the neurological deficits of I/R rats, which is statistically significant (P<0.05). This indicates that the intervention of AR113 has a certain improvement effect on the neurological deficits of I/R rats, as shown in FIG. 10(b).

### Embodiment 5: Effect of Lactobacillus plantarum AR113 on Cognitive Impairment in I/R Rats

The water maze test is one of the commonly used tools for evaluating the learning and memory abilities of rats. In the water maze test, a circular pool with a diameter of 1.6 m is set up. The movable circular platform has a diameter of 10 cm. The water level in the pool is 3 cm higher than the circular platform. The entire circular pool is divided into 4 quadrants in a clockwise direction. The movable circular platform is placed in the first quadrant. The platform search test lasts for 4 days. On the first day, the rats were put into a pool without a platform and allowed to swim freely for 2 minutes to adapt to the water environment. Starting from the second day, the platform search test is performed every day, and the circular platform is placed in a fixed position in the first quadrant for training. Put the rats on the platform for 30 s, so that they know there is a platform to escape. Then, turn the rats' heads towards the pool wall, and throw the rats successively into the water for the platform search test, starting from the first quadrant. Counting from the time when the rats enter the water, each search test lasts for 90 s. If the rat finds the platform within 90 s, the time of it is the latency for the rat to search for the platform in this quadrant. If the rat does not find the platform within 90 s, guide it to the platform, let it rest for 30 s and place it in the water again to find the platform until it finds the platform within 90 s. The time of the last successful attempt is recorded as latency. When the platform search test is done in one quadrant, the rats are placed on the platform to rest for 30 s before starting the test in the next quadrant. The average of the latency recorded in the four quadrants is the latency of the rat on that day. The rats are bred normally during the time period.

FIG. 11 is the schematic diagram of the movement track of the water maze experiment in embodiment 5. FIG. 11(a) is a trajectory diagram of the water maze test in the sham operation group. FIG. 11(b) is a trajectory diagram of the water maze test in the I/R group. FIG. 11(c) is a trajectory diagram of the water maze test in the AR113 intervention group.

The results of the water maze test showed that compared with the Sham group, the I/R injured rats had significantly longer escape latency. And the number of times they crossed the platform within a certain period of time was decreased. In the AR113 intervention group, cognitive dysfunction of rats was improved, the escape latency was shorten, and the number of platform crossings was increased, as shown in Table 6. As shown in FIG. 11, the movement patterns show the movement trajectories followed by different groups of rats. The figure clearly shows that the rats in the I/R group always swim along the wall and spent more time searching the platform. On the contrary, the rats in the AR113 group could locate the platform faster, and their performance was significantly better than the rats in the I/R group. This study shows that intraoral intervention of Lactobacillus plantarum AR113 can improve memory impairment after cerebral ischemia and reperfusion in rats.

TABLE 6
Comparison of learning and memory abilities of rats among groups

| | | | | Platform |
|---|---|---|---|---|
| | Escape latency(s) | | | crossing |
| Group | Day 1 | Day 2 | Day 3 | times |
| Sham | 28.31 ± 8.26 | 20.27 ± 3.65 | 17.88 ± 3.73 | 4.56 ± 1.35 |
| I/R | 61.12 ± 13.17* | 46.74 ± 6.76* | 39.82 ± 8.97* | 1.33 ± 1.01* |
| AR113 | 40.86 ± 10.56 | 34.36 ± 8.12 | 25.17 ± 4.59# | 2.87 ± 1.21 |

Note:
*P < 0.05 vs Sham group;
#P < 0.05 vs I/R group.

### Embodiment 6: Effect of Lactobacillus plantarum AR113 on Cerebral Infarct Volume in I/R Rats

FIG. 12 is the diagram of the effect of AR113 on cerebral infarction volume in I/R rats in embodiment 6. FIG. 12(a) is a representative image of TTC-stained brain tissues in each group. FIG. 12(b) is the ratio of cerebral infarction volume of rats in each group, wherein ***P<0.001 vs Sham group; #P<0.05 vs I/R group.

TTC (2,3,5-triphenyltetrazolium chloride) is soluble in water, but after being reduced by mitochondrial dehydrogenase of normal tissue cells, it will form a deep red fat-soluble light-sensitive component. Its staining effect is closely related to the activity of mitochondrial dehydrogenase. Therefore, with TTC staining, normal tissues are stained dark red, while avascular necrotic tissues are not stained and are pale. This is because the dehydrogenase activity is lost and TTC cannot be reduced, as shown in FIG. 12(a). The results of TTC staining showed that there was no abnormality in the sham operation group, and the brain tissues were all stained strong dark red, but obvious infarcts were seen in the I/R group. Compared with the sham operation group, the percentage of cerebral infarction volume in the I/R group increased significantly (P<0.001), while the intraoral intervention of AR113 significantly reduced the cerebral infarct volume of the I/R rats. This indicates that Lactobacillus plantarum AR113 has a certain positive effect on the reduction of rat cerebral infarction volume caused by I/R model, as shown in FIG. 12(b).

### Embodiment 7: Morphological Changes of Brain Tissues in I/R Rats

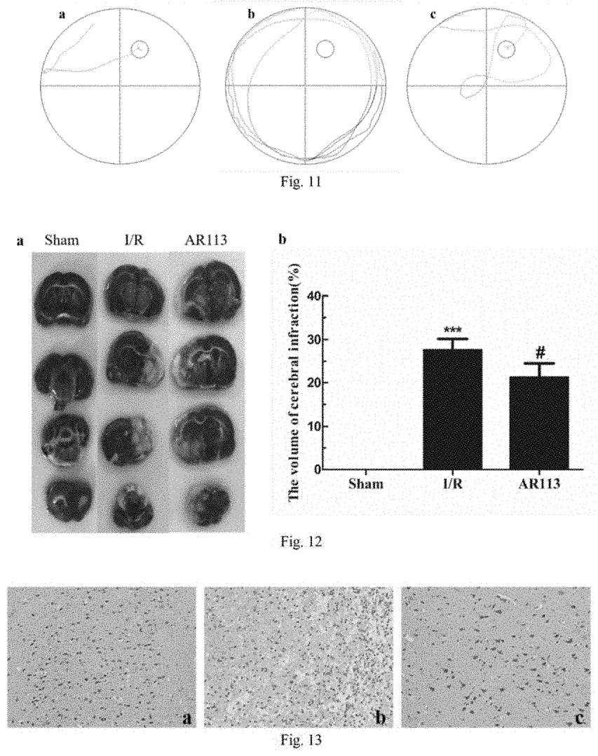

FIG. 13 is the diagram of HE-stained brain tissues (×200) in embodiment 7. FIG. 13(a) is a schematic diagram of HE-stained brain tissues in the sham operation group. FIG. 13(b) is a schematic diagram of HE-stained brain tissues in the I/R group. FIG. 13(c) is a schematic diagram of HE-stained brain tissues in the AR113 group.

Irreversible damage to nerve cells caused by cerebral ischemia and reperfusion is the main cause of learning and memory impairment. The brain tissues of rats in each group were HE-stained, and the results are shown in FIG. 13. The neuronal cell structure of rats in the sham operation group was clear under the microscope, and the cytoplasm is stained homogeneously, as shown in FIG. 13(a). The neuronal cells of rats in the I/R group are seriously injured, with inflammatory cell infiltration, a large number of red blood cells overflow and tissue edema. The color of intercellular substance becomes lighter, showing obvious pathological changes, as shown in FIG. 13(c). Compared with the model group, the intervention of AR113 significantly improved the nerve cell damage caused by cerebral ischemia and reperfusion, and maintained the integrity of brain tissues.

### Embodiment 8: Effect of Lactobacillus plantarum AR113 on Oxidative Stress in Brain Cells of I/R Rats

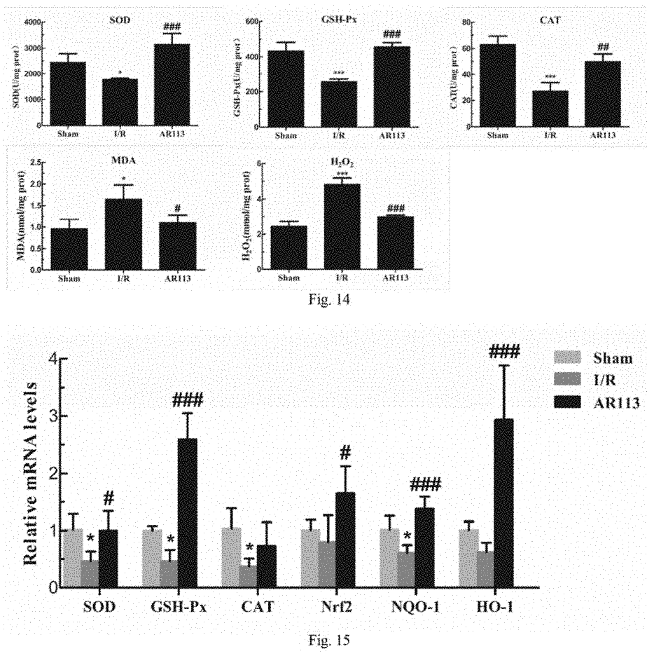

FIG. 14 is the schematic diagram of the effect of AR113 on the levels of SOD, GSH-Px, CAT enzyme activity, MDA and H₂O₂ in the brain tissues of I/R rats in embodiment 8, wherein *P<0.05, ***P<0.001 vs Sham group; #P<0.05, ##P<0.01, ###P<0.001 vs I/R group.

FIG. 15 is the schematic diagram of the regulatory effect of AR113 on the antioxidant-related factors in the brain tissues of I/R rats in embodiment 8.

Antioxidant enzyme activity and oxidation products were tested on the brain tissues of the I/R rats, and the results are shown in FIG. 14. The activities of SOD, GSH-Px and CAT in the brain tissues of rats in the I/R group were significantly lower than those in the sham operation group (P<0.05; P<0.001), while the oxidation products MDA and H₂O₂ levels increased significantly (P<0.05; P<0.001). Compared with the I/R group, after AR113 treatment, the activities of SOD, GSH-Px and CAT were significantly increased while the expression of oxidation products was decreased (MDA, P<0.05; H₂O_{2,} P<0.001). The RNA extracted from rat brain tissues was subjected to qPCR to detect the relative expression of antioxidant-related factor mRNA. The results showed that the intervention of AR113 can increase the mRNA level of Nrf-ARE pathway-related genes to varying degrees, and the antioxidant enzyme gene expression level and activity change trend are basically consistent, as shown in FIG. 15. This indicates that Lactobacillus plantarum AR113 can regulate the gene expression of key downstream antioxidant enzymes by activating the Nrf2 signaling pathway, increase the activity of antioxidant enzymes in the brain tissues of I/R rats, reduce the oxidative stress damage caused by ischemia-reperfusion, and enhance the body's antioxidant activity ability.

### Embodiment 9: Effect of Lactobacillus plantarum AR113 on Brain Cell Apoptosis in I/R Rats

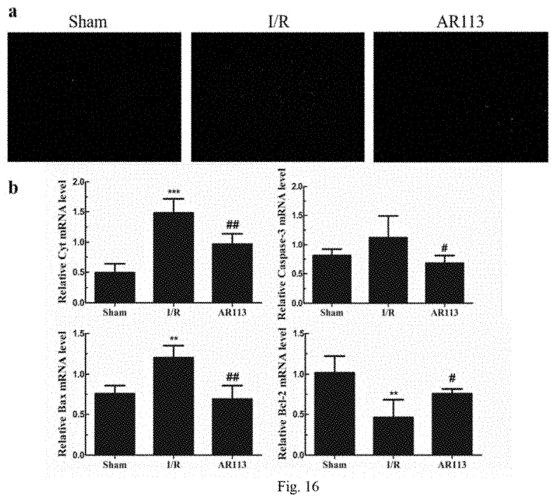

FIG. 16 is the schematic diagram of the effect of AR113 on brain cell apoptosis in I/R rats in embodiment 9. FIG. 16(a) is the schematic diagram of mRNA levels of apoptosis-related factors. FIG. 16(b) is the schematic diagram of TUNEL-stained brain tissues (×200), wherein **P<0.01, ***P<0.001 vs Sham group; #P<0.05, ##P<0.01 vs I/R group.

Secondary neuronal apoptosis may be an important pathological basis for cerebral ischemia-reperfusion injury, and it is also the main manifestation of cerebral ischemia-reperfusion injury. Apoptosis is the main way of cell damage in ischemic penumbra area. The part of the body damage can be avoided if reasonable measures are taken. TUNEL staining is a method to detect cell apoptosis. When apoptosis occurs, the genomic DNA is broken, and the exposed 3′-OH is catalyzed by terminal deoxynucleotidyl transferase and fluorescein-labeled dUTP is added, so that is can be observed through a fluorescence microscope. Perform TUNEL staining on the brain tissues of I/R rats to observe the apoptosis of nerve cells (positive cells are fluorescent green). The result is shown in FIG. 16(a). Compared with the sham operation group, the number of apoptotic cells in the brain tissue of rats in the I/R group was significantly increased. While the status of apoptotic cells in the AR113 group was significantly improved compared with the I/R group.

Changes of mitochondrial respiratory chain are the main direction of current apoptosis research. Cytochrome Cyt-C is a basic component in the respiratory chain, and it is also the initiating factor for mitochondria to regulate cell apoptosis. Cyt-C is released from the mitochondria to the cytoplasm and activates Caspase, triggering a cascade reaction, leading to cell apoptosis. The Bcl-2 protein family can regulate the release of Cyt-C. Pro-apoptotic gene Bax and anti-apoptotic gene Bcl-2 regulate the release of Cyt-C by regulating mitochondrial membrane channels. The relative mRNA expression of apoptosis-related genes was detected by qPCR experiment. As shown in FIG. 16(b), the mRNA levels of the pro-apoptotic factors Cyt, Caspase-3 and Bax in the brain tissues of I/R rats were up-regulated compared with the sham operation group, wherein the increase in Cyt and Bax mRNA levels is statistically significant (P<0.001; P<0.01). The mRNA level of inhibitory factor Bcl-2 decreased significantly in the I/R group (P<0.01), indicating that the I/R model induced by MCAO can cause rat brain cell apoptosis and aggravate cell damage. Compared with the model group, AR113 intervention can significantly down-regulate the mRNA levels of pro-apoptotic factors (Cyt, P<0.01; Caspase-3, P<0.05; Bax, P<0.01), and up-regulate the anti-apoptotic factor Bcl-2 MRNA expression (P<0.05). The result shows that AR113 intervention can inhibit cell apoptosis caused by cerebral ischemia and reperfusion, thereby exerting a protective effect on its injury.

According to above embodiments, a rat focal cerebral ischemia model was prepared using middle cerebral artery occlusion (MCAO), and an I/R model was prepared after 2 hours of ischemia and reperfusion. They were used to explore whether intraoral application of probiotics has a protective effect on cerebral ischemia-reperfusion injury in rats and its possible mechanism, so as to obtain effective data and provide a theoretical basis for the intervention and treatment of probiotics for apoplexy.

In addition, it can be seen from the above embodiments that the intervention of Lactobacillus plantarum AR113 in the intraoral cavity of cerebral ischemia-reperfusion (I/R) model rats can significantly regulate the imbalance of the tongue bacterial flora of I/R rats and improve the degree of brain damage. It also has a regulating effect on intestinal flora of I/R rats. In addition, the administration of AR113 can significantly improve neurological deficits in I/R rats and improve the ability of learning and memory. The present invention shows that AR113 can activate the Nrf-ARE signal pathway by increasing the antioxidant enzyme activity of brain tissue, reducing the level of oxidized products, regulating the relative expression of antioxidant factors Nrf2, NQO-1, and HO-1, and at the same time down-regulating the mRNA expression of pro-apoptotic factors Cyt-C, Caspase-3 and Bax, up-regulating the relative expression of the anti-apoptotic factor Bcl-2, thereby improving brain cell damage caused by cerebral ischemia and reperfusion.

In summary, the application of the Lactobacillus plantarum AR113 of the present invention in the intraoral cavity of I/R rats has a regulatory effect on its tongue bacterial flora and intestinal flora, and has a certain protective effect on neurological deficits and brain damage. The main reason is that AR113 has excellent antioxidant and anti-apoptotic activities. Intraoral application of AR113 can regulate the imbalance of the tongue bacterial flora caused by I/R injury, and can maintain the steady state of intestinal flora. At the same time, AR113 can alleviate neurological deficits and brain cell oxidative stress damage caused by I/R injury and have a certain improving effect on apoptosis.

1. A Lactobacillus plantarum strain AR113 with a protection effect on apoplexy, said Lactobacillus plantarum strain AR113 deposited in China General Microbiological Culture Collection Center (CGMCC) under CGMCC Accession No. 13909 on Mar. 22, 2017.

2. A product comprising said Lactobacillus plantarum strain of claim 1 to provide the protection effect on apoplexy.
